(19) United States
(12) Patent Application Publication
(10) Pub. No.: US 2015/0057621 A1
(43) Pub. Date: Feb. 26, 2015

(54) Headgear

(71) Applicant: Snowraft Pty Ltd, Cashmere (AU)

(72) Inventor: Clyde Kevin Coombs, Cashmere (AU)

(21) Appl. No.: 14/267,016

(22) Filed: May 1, 2014

(30) Foreign Application Priority Data
Aug. 26, 2013 (AU) AU 2013220865
Mar. 4, 2014 (AU) AU 2014201165

(51) Int. Cl. A42B 1/18; A61M 35/00

(52) U.S. Cl. CPC A42B 1/18; A61M 35/00
USPC 604/289; 2/424

(57) ABSTRACT

This invention relates to headgear for help in protecting the wearer against facial injury, especially skin damage from the elements and in particular sunburn and windburn, the headgear comprising a fabric wall which substantially surrounds the face and neck of the wearer and has in it an aperture for locating adjacent the wearer's eyes to allow vision. The wall is lightweight, flexible and porous and has stiffening means at the periphery of the aperture so as to support the aperture in open configuration in a vision-facilitating position adjacent the eyes of a wearer in use, and the wall provides protection to the wearer against ingress of particulate projectiles from around the transparent first membrane, as well as a degree of shielding from UV radiation while remaining lightweight and breathable.

### FIELD OF INVENTION

This application claims priority to Australian application no. AU 2014201165, filed on Mar. 4, 2014, invented by Clyde Kevin Coombs and filed by Snowraft Pty Ltd, which is incorporated herein by reference in its entirety.

### PRIORITY CLAIM

This invention relates to headgear for protecting the wearer's head and neck against exposure to the elements and in particular the effects of sunburn and windburn.

### BACKGROUND TO THE INVENTION

Many forms of headgear exist. Apart from traditional head-dress forms associated for example with persons of certain Arabic cultures, and religious orders, (Christian nun habits and Islamic burqas) there are turbans, helmets such as soft balaclava helmets, and hard impact resistant biking helmets, skier helmets, cricket and football helmets and military helmets, including the pith helmet, as well as fire-proof hoods. These have in common a covering for the wearers head and at least a portion of the neck.

A drawback of the above forms of headgear is their specialised purpose, usually making them impractical to use in other applications, in which particular feature or characteristic is desirable, but not other features.

Another drawback arising from certain of the above types of headgear, is the cultural and religious associations they evoke.

### OBJECTS OF THE INVENTION

It is an object of this invention to address the shortcomings of the prior art and, in doing so, to provide an item of headgear that is suitable for use in outdoor activities in which prolonged exposure of the wearer to any one or more of heat, sun and wind is anticipated.

A further object of the invention is to provide means for reducing the risk to the wearer of sunburn, windburn and heat-exhaustion.

Another object is to provide protective headgear that is low-cost and low-tech, making it suitable for cheap mass production and ready availability.

The preceding discussion of the background to the invention is intended to facilitate an understanding of the present invention. However, it should be appreciated that the discussion is not an acknowledgement or admission that any of the material referred to was part of the common general knowledge in Australia or elsewhere as at the priority date of the present application.

Further, and unless the context clearly requires otherwise, throughout the description and the claims, the words ‘comprise’, ‘comprising’, and the like are to be construed in an inclusive sense—that is to say, in the sense of “including, but not being limited to”—as opposed to an exclusive or exhaustive sense—that is to say meaning “including this and nothing else”.

### SUMMARY OF INVENTION

According to the invention, there is provided protective headgear suitable for outdoor use, the headgear comprising a head-receiving cavity and a wall of light-weight UV radiation-disrupting fabric, configured to be downwardly depending when worn by an upstanding wearer and encircle the wearer's neck, the wall having a vision-enabling aperture located to be positioned before said wearer's eyes and comprising means for preventing closure of said aperture.

In a preferred form of the invention, the aperture and wall are configured not to expose the wearer's lips.

Further according to the invention, the wall preferably comprises a textile. An example is a woven cloth.

Further preferably, the wall is resistant to ingress of water. (providing protection against salt-sea spray).

The fabric is preferably selected from textiles having a ultraviolet protection factor (UPF) of at least 20. (A UPF rating of 20 indicates the fabric of a garment will allow only 1/20th (roughly 5%) of available UV radiation to pass through it). Further preferably the textile UPF is 50 or greater.

In a preferred form of the invention, the headgear further comprises means for supporting the wall so that the vision-enabling aperture is maintainable in vision-facilitating position adjacent the eyes of a wearer in use

But for the vision-enabling aperture adjacent the wearer's face, the wall provides protection to the wearer against ingress of particulate matter, excessive wind and ultra-violet radiation. It is loose fitting and able to dissipate the momentum of particles striking it.

In an embodiment of the invention, the the closure-preventing means comprises stiffening means positioned proximate an edge of said aperture. In preferred embodiments, the stiffening means substantially surrounds the cavity.

In a preferred embodiment of the invention, the wall defines the cavity.

In an alternative embodiment, the cavity is defined by headwear to which the wall is attached. Preferably, the wall is removably fastenable to the headwear.

Further, in this embodiment, the headwear comprises a hat or a cap.

Preferably, the wall is at least partly retractable into the cavity for retention therein when not in use.

Further, according to the invention, the wall comprises aperture supporting means for supporting the aperture before the wearers eyes.

The supporting means preferably comprises adjustment means for allowing adjustable fitment of the headgear to the wearer's head.

In an embodiment, the supporting means comprises elastic band means, having elasticity sufficient for achieving a snug fit around wearer's head.

In a further preferred form of the invention, the wall comprises a second, breathing aperture, positioned to be locatable adjacent the wearer's mouth in use. The breathing aperture is separated from the vision-enabling aperture.

In an embodiment, the second aperture is covered by a breathable membrane. In a further preferred embodiment, the breathable membrane comprises fabric meshing. In an example, the membrane comprises a mesh, such as a mosquito-excluding gauze.

In a further embodiment, the membrane is displaceable in use.

In a further preferred form of the invention, the wall comprises fastening means for fastening the breathable membrane to the wall in a position for covering said breathing aperture in use.

In a preferred embodiment, the fastening means comprises stitching.

Preferably, the fastening means fastens an upper peripheral portion of the breathable membrane to the wall at a location above the breathing aperture only, so that the breathable membrane depends downwardly to cover the breathing aperture when in use. Said upper peripheral portion, in an embodiment, is a strip of the breathable membrane located adjacent the wall.

In a preferred form, the breathable membrane defines a displaceable flap which can be lowered or raised according to wearer preference. Releasable secural means may be provided for securing a lower peripheral edge of the flap to an adjacent portion of the wall when in operative depending position. Releaseable secural is achievable by providing complemental hook-and-loop closure means operatively located on the breathable membrane and the wall.

The invention extends optionally to the headgear having fastening means fastening a side peripheral portion of the breathable membrane to the wall proximate to a side of the breathing aperture. In an embodiment, the fastening means comprises stitching.

Still further, according to the invention, the headgear comprises supporting means for the supporting the aperture in operative orientation before the wearer's eyes.

In a further preferred embodiment, the supporting means comprises adjustment means for allowing adjustable fitment of the headgear to the wearer's head.

Thus the supporting means comprises a component adapted for encirclement of the wearer's head at a position above the wearer's eyes. It extends around the head of the wearer in use to be supported thereby, and supports the wall to hang down to the neck of the wearer.

Preferably, the band means has an adjustable functional length. Preferably, said component is tensioned against the wearer's head in use, to resist displacement therefrom. The component may comprise elongate band means.

Preferably, in this form of the invention, the wall further comprises a sleeve formed above the level of the vision aperture when in operative orientation and into which sleeve the band means is inserted to at least be partly received and contained.

In a particularly preferred form of the invention, the supporting means comprises elastic band means, having elasticity sufficient for achieving a snug fit around wearer's head.

In an embodiment, the headgear comprises a plurality of hoops connected to the wall to define a guideline through which the band means is passed in operative supporting orientation.

In an embodiment, the band means comprises adjustable belt means. Preferably it comprises an elongate strip with mutually engageable opposed ends.

In another embodiment, the band means comprises drawstring means. Alternatively, the band means is at least in part elastic. In an embodiment, the band means is connected to the wall.

The wall optionally comprises a ventilation slit. In such an embodiment, the ventilation slit is preferably generally vertically disposed and may extends for a part of the length of the wall. Preferably the slit is located rearward of the wearer's ears when the headgear is worn in use.

Further, according to the invention, the supporting means comprises a hood portion of the wall and extends over the head of the wearer to rest thereon in use.

Further preferably, the hood portion comprises a crown panel that is integrally formed with the wall. The hood portion in use is at least partly supported by the head of the wearer.

In another form of the invention, the crown panel is integrally formed with a brim portion that extends generally radially from the wearer's head.

In an embodiment of the invention in an alternative form, the wall is connected to a brimmed hat to depend downwardly therefrom in use and be retractable into a cavity defined by the hat when the wall is not required for protective purposes. Preferably, the hat is of self-supporting material.

In a still further preferred form of the invention, the headgear wall defines a lower end in use and has orientation-assisting means proximate said lower end, said orientation-assisting means being arranged to urge said lower end to a position of proximity to the wearer's neckline. In such arrangement, the headgear is adapted for inhibiting ingress of particulate projectiles to internal space defined by the headgear and adapted against billowing up over the wearer's face in windy conditions.

In an embodiment, the orientation-assisting means comprises second band means. Preferably, a lower periphery of the wall comprises a hem within which the band extends and is retained.

In an alternative embodiment, the orientation-assisting means comprises weighting means contained in the hem.

In a yet further form of the invention, the wall additionally comprises a pocket. Preferably, the pocket is located at a lower, side portion of the wall. Further preferably, it is located above and proximate to the hem.

In an embodiment, the pocket is dimensionally adapted to contain a sachet, preferably charged with a functional substance. Preferably, the sachet contemplated contains a pharmaceutical.

The pharmaceutical may comprise a cosmetic. The cosmetic in an example comprises a sunscreen preparation (for application, for example, to exposed portions of the wearer's face surrounding the wearer's eyes). In another example, the cosmetic is a skin moisturiser. In a further example the sachet contains an eye-care preparation.

In an alternative embodiment, the sachet contains a dietary supplement. Preferably the supplement comprises a formulation adapted to provide replenishment to the headgear wearer according to an expected activity of the wearer when using the headgear. For example, if the wearer is expected to engage in an activity under conditions likely to result in profuse perspiration, the sachet will contain a formulation comprising tissue salts in a rehydrating carrier gel. Known formulations of this nature are available from specialist nutrition providers.

Preferably, the headgear further comprises a hanging-eye attached to the wall. The hanging-eye in an embodiment is attached to an apical region of the wall. In an alternative embodiment it is attached proximate a lower end of the wall, preferably at its rear when worn in use.

### BRIEF DESCRIPTION OF DRAWINGS

In order that the invention may be readily understood, and put into practical effect, reference will now be made to the accompanying figures. Thus:

FIG. 1 shows front, rear, side and top views of a preferred embodiment of the headgear of this invention.

FIG. 2 is a front view of an alternative, embodiment of the headgear of the invention.

FIG. 3 is a front view of a further alternative embodiment of the inventive headgear.

FIG. 4 is a partially exploded front perspective view of a further embodiment of the headgear of this invention.

### DETAILED DESCRIPTION OF EMBODIMENTS OF THE INVENTION

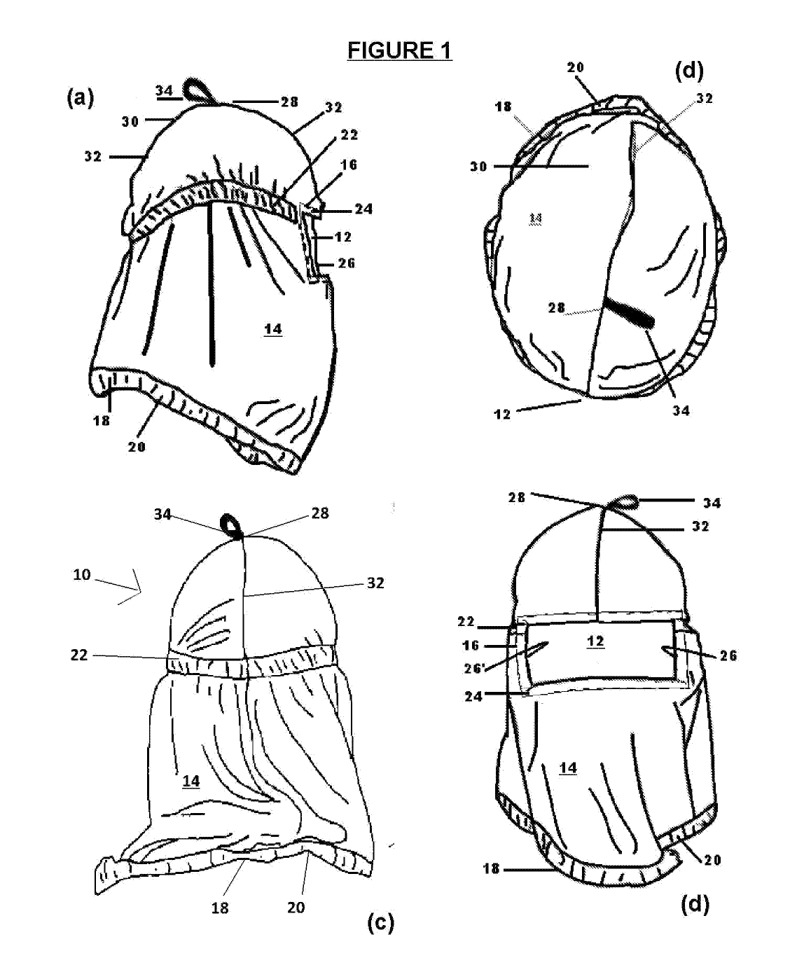

Referring to FIG. 1, an item of headgear, according to a preferred embodiment of this invention, is generally denoted by the number 10. It is configured as a hood made from a single panel of water-resistant, UV-disrupting woven fabric, defining a textile wall 14. When worn in use, the wall hangs loosely downwardly from the crown of the wearer's head (at 28), covering face and neck (not shown), except for a portion of the face exposed by an aperture 12, to be described in more detail below.

A seam 32 extends over apex 28 of the headgear and down the rear 30, to provide closure for wall 14 to form a hood. In other embodiments, the hood structure may be fabricated from two more panels of flexible material without departing from the scope of this invention.

An eye 34 of a looped strip of textile tape material is attached at the apical region to facilitate hanging of the hood on a suitable peg or nail when not in use

An slot-like aperture 12 is provided in the wall. As suggested by seam 16, which surrounds it, the wall fabric surrounding the slot is folded back into a hem which defines opposing sets of sleeves on either side of the aperture. Into these sleeves, stiffening ribs 24 of polyethylene are sewn. These provide a means of stiffening and stabilising the wall fabric adjacent the periphery of the aperture. The result is a reduction in any tendency of the periphery of the aperture to flap against the face of the wearer, and interfere with the wearer's field of peripheral vision through the aperture.

In alternative embodiments of the headgear of this invention, the stiffening ribs 24 may be secured in place by attachment means comprising bonding means, such as thermal bonding or by an adhesive.

Fabric wall 14 extends downwardly to cover the face and neck of the wearer. To minimise gaps between the fabric wall and the body of the wearer at its lower periphery, a hem 18 is formed there and an elastic strip 20 is fitted into it This arrangement inhibits ingress of substantial amounts of dust, sea spray or fumes from below. The length and strength of the elastic strip are selected to achieve a relatively loose, but gap-closing fit on the shoulders and neckline of the wearer. The elasticised hem helps prevent the hem or other parts of the wall membrane from snagging. In this embodiment, the wall membrane is a performance mesh of a kind available from Australian Fabric Wholesales of Mount Waverley, Victoria, in Australia. This particular mesh is 100% polyester of quality ST06. It has a gram weight of 170 gsm and yield of 3.6 mkg. It is designed for rapid moisture evaporation and to promote passage of air through it and to the skin of the wearer. This material is rated UPF:40, which means it falls into the UPF “excellent protection” category of the Australian Radiation Protection and Nuclear Safety Agency.

The vision aperture is supported in vision-enabling position by an elastic strip 22 that is sewn into wall 14 to extend around the wearer's head from one edge 26 of the aperture to the opposing edge 26′. Unlike elastic strip 20, elastic strip 22 is selected to be of sufficient tightness and length to retain aperture 12 in vision-enabling position, for all but the most violent of head movements by the wearer.

The pull exerted by the respective opposite ends of elastic strip 22 on edges 26 and 26′ of aperture 12, to which they are attached by stitching, keeps the aperture laterally stretched and disinclined to fold closed or crease to any degree that would impede vision unduly.

When the headgear is worn correctly, elastic strip 22 is positioned above the eyes and preferably around the lower part of the forehead or brow of the wearer. This positioning helps to avoid wearer discomfort resulting from undue pressure being exerted against any additional eye-wear worn by the wearer—for example prescription eyeglasses, sunglasses and protective lenses. The aperture may be dimensioned so that its lower edge is located above the line of, so as to have the wall cover, the wearer's lips. It may be dimensioned so that the lower edge passes just below, or even across the wearer's nose, in the latter case providing protection to a lower portion of the nose, such as the lowest third thereof.

Preferably, the dimensions of the aperture are selected so that the ratio width:height exceeds 0.8 but is less than about 8.0. This is found to assist in inhibiting lateral obstruction by discouraging laterally extending concavities/ convexities from developing in the textile proximate the edges 26, 26′. In the embodiment shown in FIG. 1, the ratio is approximately 2.5.

It is found that by keeping the lateral dimension (width) of aperture 12 in the range from 120 mm to 200 mm, unacceptable flapping or distortion of its shape by tending to close is largely avoided. It is desirable that the width exceed the distance from eye to eye of the wearer, enabling a degree of exposure to the face to the sides of the eyes to prevail. This effect helps to prevent lateral and vertical distortion of the vision aperture, as well as increasing the user's field of peripheral vision.

Although shown as being a slot of generally rectangular shape, the aperture may advantageously have rounded corners or even be an oval in the alternative. Other shapes that accommodate the features of a user's eyes and nose may also be utilised, for example the shape of “aviator” style sunglasses, for example that of the Porsche Carrera® sunglasses. It is not beyond the scope of this invention for the headgear to provide separate first and second vision-enabling apertures.

Referring to FIGS. 1(c) and 1(d), rear and top views of the same embodiment are depicted. These show how wall membrane 14 encloses the entire head and neck of the wearer. This prevents many kinds of potentially harmful particles produced in workshop-type environments, or even in garden work, from striking the bare flesh of the wearer, whether by direct trajectory from a work-piece or by a ricochet. The headgear in this embodiment therefore provides protection when the user is engaged particularly in operating power tools, such as circular saws, grinders, lathes, sanders and the like. It also provides protection in outdoor activities such as lawn mowing, hedge trimming, line trimming with the wall because of its looseness and flexibility serving to catch or deflect many particles or reduce or dissipate their momentum.

The material of the wall membrane attached around the vision aperture is selected for “breathability”, that is the capacity for air to permeate to the wearer within. Desirably, it should also absorb moisture from perspiration readily and promote its evaporation for cooling and comfort. Preferably, the material is suitably porous and permeable to enable the user to breathe in comfort while wearing the headgear in complete hood configuration.

The wall membrane is also desirably treated to be at least fire-retardant, if not completely fire-proof.

It will be appreciated that instead of having an elastic strip 22 to maintain and support the position of the vision aperture in operative orientation, as contemplated in this invention, one may alternatively have an adjustable strap that the wearer can adjust to his (or her) preference. An example of such a strap is a plastic strip in two pads, each being attached proximate to the edges 26 and 26′ of the vision aperture, and at their opposite ends having adjustable fastening means, such as a set of projecting studs on the part attached at edge 24 and a set of correspondingly positioned mating apertures or depressions in the second part attached to edge 26. Depending on the size of the wearer's head circumference, the appropriate stud can be mated with the aperture whose position corresponds with the optimum level of comfort for the wearer. Other suitable fastening means are known in the art and include releasable and adjustable locking buckles of various configurations.

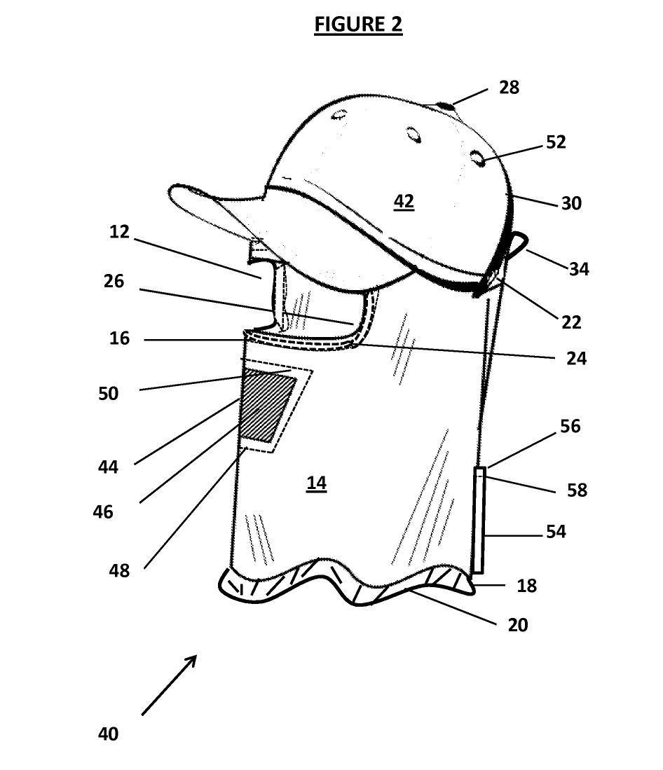

A second embodiment of the headgear of this invention, illustrated in FIG. 2 and designated by means of the general reference numeral 40, provides for a fabric wall 14, having vision enabling aperture 12, to be fitted within a cap 42 of conventional “baseball” design style. Like numbering is applied to like parts already encountered in FIG. 1.

A second aperture 44, covered by breathable gauze mesh 46, is provided to be located below the first aperture 12 and introduces specific ventilation means to facilitate the wearer's breathing under appropriate conditions. Aperture 44 is positioned to be located adjacent the wearer's mouth when in use. The aperture is shown as being generally rectangular, but may of course be of any other suitable shape, such as oval or spherical. Irregular or more exotic or decorative forms are also within the scope of this invention. Exemplary, non-limiting dimensions of the rectangular aperture in the illustration are width 50 mm by 20 mm vertical length.

Breathable sheet 46 is of a breathable fabric meshing, in this embodiment mosquito polyester gauze. The meshing overlaps aperture 44 so that it is glued at a peripheral strip 48 to the outside of surrounding wall 14. Preferably, as shown in FIG. 2, the meshing is secured to the wall along substantially the entire extent of the periphery of aperture 44, along a margin 48. However, it is to be understood that it is advantageous in certain environments to have a portion of the periphery unsecured, to allow for completely unobstructed access to the wearers mouth and nose. For example, in very hot environments or confined spaces, the wearer may wish to take a replenishing drink without having to remove the headgear. For this situation, the meshing sheet is secured by stitching it to wall 14 in only that part of peripheral margin strip 48 that lies adjacent the upper periphery of aperture 44, as denoted by the numeral 50. Optionally, the meshing sheet is disengageably secured adjacent its lower, opposite peripheral edge by way of a coupling such as a button, hook-and-loop (Velcro®) or press-stud, so that the meshing then functions as a flap, but does not flap upwards to obscure vision through vision aperture 12, when operative.

In certain severely dusty environments, or where copious quantities of very small particulate projectiles (smaller than the mesh rating covering aperture 44) are being ejected from a work-piece, it may not be desirable to provide an aperture of the kind shown in FIG. 2, and the embodiment described with reference to FIG. 1 may be more desirable. Thus it will be appropriate when activities are expected to be performed under certain types of conditions, for the headgear to be provided with a wall material selected for greater or less ease of “breathabilty” (the ease with which a wearer is able to breathe though the wall itself). For some conditions, a denser wall material may be paired with a breathable meshing covering a breathing aperture, while in others, a very light wall material of low density and greater breathability may be selected. In a further enhancement of this embodiment, there is provided a press-stud on the outer lower portion of the depending flap membrane and a complemental stud-receiving female base formation located above and between the breathing aperture and the vision aperture (not shown in the figures), enabling the flap to be raised and releaseably secured in a raised position at the wearer's discretion. When so raised, the wearer may be better able to speak or to eat or receive a beverage than when the flap is lowered. Instead of a press-stud pair, temporary secural may be achieved by other fastening means well known in the art, for example hook-and-loop connectors and buttons.

Headgear 40 has additional ventilation apertures 52 provided in the upper portion of cap 42.

An optional item illustrated in the embodiment of FIG. 2 is a side pocket 54, accessible from the exterior of the headgear via opening 56. The pocket is shown located at a lower, side portion of the wall toward the rear of the headgear. However, it may be positioned elsewhere in other embodiments, according to decorative or styling requirements, as well as or in addition to function.

In FIG. 2, pocket 56 is located above and proximate to hem 18. The pocket is shown comprising a fabric overlay sewn on three sides to the wall and being open at a hem 58 at its upper edge. The overlay is made of the same fabric as wall 14, but need not be so.

In another embodiment, the pocket is in the form of a pouch located on the inside of the headgear and accessible from the outside via a suitable slit in wall 14 where hem 58 is located. In this case the internal pocket is not of the same fabric, but is for example nylon, or a nylon/cotton blend.

The pocket is suitably sized to hold a sachet containing a functional product useful to the wearer and the task being carried out using the headgear. In the example of FIG. 2, the sachet contains a sun-screen composition of known formulation and in a quantity sufficient for application to the face area visible through the vision-enabling aperture (12).

The pocket may be sized to hold two or more sachets of functional substances. For example, alternatively or additionally, the pocket may contain a sachet having in it a pharmaceutical, such as an eye-rehydrating formulation (e.g. for use when the wearer has been exposed to wind burning conditions during an outdoor activity). In another example, the cosmetic is a skin moisturiser for application in dry and/or cold environments. In a further example the sachet contains a lip balm. In another, the sachet contains a nutritional gel containing rehydrating electrolytes.

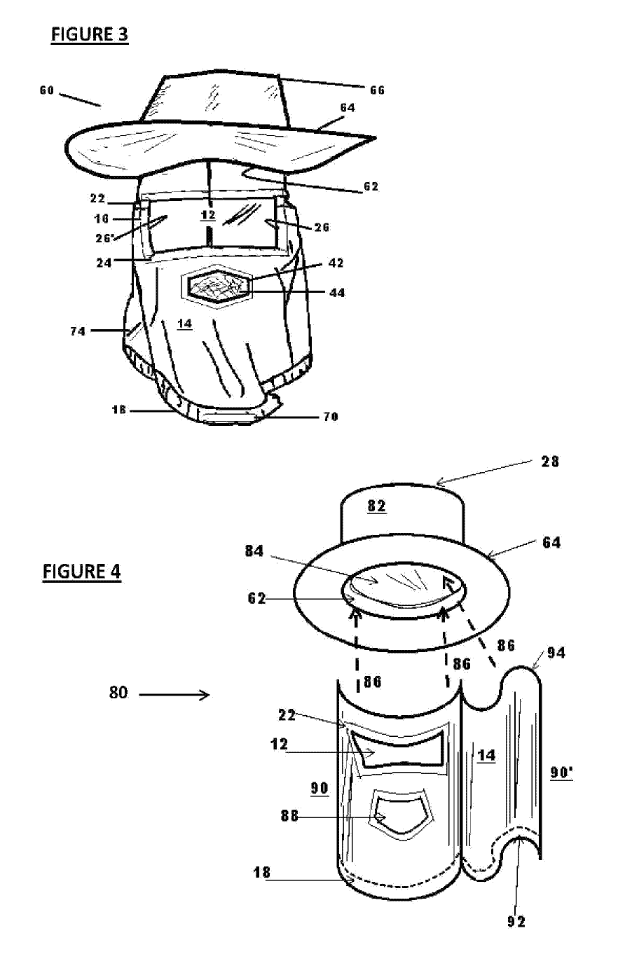

FIG. 3 shows the front of a further embodiment of the invention. Here headgear 60 comprises a wall 14 depending from and attached to the inner lining 62 of a field hat of generally known design, having an apical portion 66, which defines a head-receiving cavity. To render the wall removable from the hat for purposes such as cleaning, it is fastened in place by complemental hook-and-loop attachment means provided respectively on the inner lining 62 of the hat cavity and the outer surface of the wall, proximate its upper end that fits against a corresponding cavity lip.

The field hat has a brim 64, which in use will cast some shade upon portions of wall 14 below it, and thereby providing some relief from direct radiative heating from the sun. Below the brim and located in fabric wall 14 are a first vision-enabling aperture 12 and a second, breathing aperture 44 covered by a mosquito gauze meshing 46.

Above the brim, there is an apical portion 66, defining an internal cavity. The cavity is sized to be able to receive the wall when retracted or pushed up into it, as well as the portion of the wearer's head normally received into it in use. It is desirable for the hat, when placed on the head of a wearer, to be worn without the wall or a significant part of it necessarily protruding. The cavity thus provides a facility for the storage of the wall when not required for use.

At the lower extremity of wall 14 is a hem 18 into which are sewn elongate strips 70 of high density polyethylene (HDPE) to serve as weights for weighing down the wall into a suitably protective orientation. An internal pocket marked by access slit 74 is provided for storing small items, for example one or more sachets of functional substances, as previously described, to be readily accessible by the wearer of the headgear.

In an example of another embodiment of the invention, the length of the depending wall below the vision aperture is open to some variation, the shorter the length, the greater the likelihood that a portion of the lower hem (18 in the drawings) will mostly hand free from contact with the body of the wearer, leaving a gap for ingress of cooling drafts. However, the minimum desirable length as measured from the horizontal midline of the vision-enabling aperture to the bottom of hem 18, is 14 cm, to result in the wall extending in front to below the chin of an adult male wearer of average proportions, and to rest on or below the collar of any collared shirt being worn. Preferably, the length as so measured should exceed 18 cm, and further preferably 21 cm.

In preferred embodiments, the wall is longer in the front than at the rear of the headgear, so as to compensate for the generally longer area of exposure of a wearer at his throat and neckline in front. The difference in lengths of the front compared with rear portion according to the measurement method above is preferably in the range 2-20 cm, and more preferably in the range from 2.5 cm to 18.0 cm. Even more preferably, the difference is in the range 9 cm to 12 cm.

In FIG. 4 is shown a still further embodiment, 80, wherein a hat 82 of generally known configuration, having an apical end 28, and a brim 64, between which are defined an internal cavity 84, for receiving part of the wearer's head in a snug fit. Wall 14, of light, but UV-resistant fabric, is shown ready for positioning by directional arrows 86, and attachment, by stitching, to inner lining band 62 within hat cavity 84.

As in previous embodiments, there is a vision-enabling aperture 12, located in the forward-facing wall portion for positioning before the wearer's eyes in use, its edges stiffened by insertion of low density polyethylene strips into the surrounding hem. Below is a second, breathing aperture, 88. In this embodiment, unlike in those described previously, this aperture is left uncovered. Opposed side edges 90 and 90′ of wall 14 are stitched together to provide a continuous wall for encirclement of the wearer's head and neck. Lower edge 92 of wall 14 is longer than the upper edge 94, which it sewn into hat 82, providing a generally conical or flared shape to the headgear, so that the wall splays over the wearer's shoulders in use, avoiding close clinging about the wearer's neck. This helps to facilitate excellent ventilation for effective cooling, while maintaining sun and wind protection. Hem 18 is gently weighted with internal strips of high-density polyethylene, to mitigate excessive flapping.

These embodiments merely illustrate particular examples of the headgear product of the invention. With the insight gained from this disclosure, the person skilled in the art is well placed to discern further embodiments by means of which to put the claimed invention into practice.

1. Headgear comprising: a head-receiving cavity and a wall of light-weight UV-radiation-inhibiting fabric; said headgear configured for the wall to be downwardly depending when worn by an upstanding wearer and encircle the wearer's neck; the wall having a vision-enabling aperture located to be positioned before said wearer's eyes and comprising means for preventing closure of said aperture.

2. Headgear of claim 1 wherein the aperture is configured not to expose the wearer's lips.

3. Headgear of claim 1 wherein the fabric is resistant to water ingress.

4. Headgear of claim 1 wherein the fabric comprises a textile selected from textiles having a UPF rating of at least 20.

5. Headgear of claim 4 wherein the textile UPF rating is at least 50.

6. Headgear of claim 1 wherein the closure preventing means comprises stiffening means positioned proximate an edge of said aperture.

7. Headgear of claim 6 wherein the stiffening means substantially surrounds the cavity.

8. Headgear according to claim 1 wherein the wall defines the cavity.

9. Headgear according to claim 1 wherein the cavity is defined by headwear to which the wall is fastened.

10. Headgear of claim 9 wherein the wall is removably fastenable to the headwear.

11. Headgear of claim 9 wherein the headwear comprises a hat or a cap.

12. Headgear according to claim 9 wherein the wall is at least partly retractable into the cavity for retaining or storing therein when not in use.

13. Headgear according to claim 1 wherein the wall comprises aperture supporting means for supporting the aperture in operative orientation before the wearer's eyes.

14. Headgear of claim 13 wherein the supporting means comprises adjustment means for allowing adjustable fitment of the headgear to the wearer's head.

15. Headgear of claim 14 wherein the supporting means comprises elastic band means, having elasticity sufficient for achieving a snug fit around wearer's head.

16. Headgear according to claim 1 wherein the wall comprises a second aperture for breathing through, being positioned to be locatable adjacent the wearer's mouth in use.

17. Headgear of claim 16 wherein the second aperture is covered by a breathable membrane.

18. Headgear of claim 17 wherein the breathable membrane comprises a mesh.

19. Headgear according to claim 16 wherein the vision-enabling aperture is separated from the second aperture.

20. Headgear according to claim 1 wherein the wall defines a lower end whereat it comprises an elasticised hem.

21. Headgear of claim 20 wherein the wall comprises weighting means proximate the lower end in use.

22. Headgear of claim 21 wherein the weighting means is contained in said hem.

23. Headgear according to claim 1 wherein the wall comprises a pocket.

24. Headgear of claim 23 wherein the pocket is dimensionally adapted to contain a sachet.

25. Headgear of claim 24 further comprising a sachet adapted to be placed in the pocket.

26. Headgear of claim 25 wherein the sachet contains a pharmaceutical.

27. Headgear of claim 26 wherein the pharmaceutical has sun-screening properties.

28. Headgear of claim 26 wherein the sachet contains a dietary supplement.
